(19) United States
(12) Patent Application Publication
(10) Pub. No.: US 2010/0167924 A1
(43) Pub. Date: Jul. 1, 2010

(54) Azolylmethyloxiranes, Their Use for Controlling Phytopathogenic Fungi and Compositions Comprising Them

(75) Inventors: Jochen Dietz, Mannheim (DE); Thomas Grote, Wachenheim (DE); Bernd Müller, Frankenthal (DE); Jan Klaas Lohmann, Ludwigshafen (DE); Jens Renner, Bad Dürkheim (DE); Sarah Ulmschneider, Bad Dürkheim (DE); Alice Glättli, Mannheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/305,730

(22) PCT Filed: Jun. 14, 2007
(86) PCT No.: PCT/EP2007/055870

§ 371 Date: Dec. 19, 2008

(30) Foreign Application Priority Data
Jun. 23, 2006 (EP) 06115929.9

(51) Int. Cl. C07D 405/06; A01C 1/06; A01N 43/653; A01P 3/00

(52) U.S. Cl. 504/100; 548/267.8; 514/383

(57) ABSTRACT

The present invention relates to azolylmethyloxiranes of the general formula I

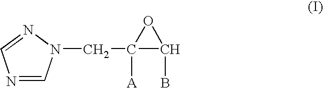

in which
A is phenyl which is substituted by three F,
B is unsubstituted pyridyl, thienyl, thiazolyl, oxazolyl or furyl or is phenyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄alkylamino, C₁-C₄-dialkylamino, thio or C₁-C₄-alkylthio,
   and to the plant-compatible acid addition salts or metal salts thereof, to the use of the compounds of the formula I for controlling phytopathogenic fungi and to compositions comprising these compounds.

The present invention relates to azolylmethyloxiranes of the general formula I

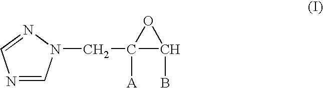

in which
A is phenyl which is substituted by three F,
B is unsubstituted pyridyl, thienyl, thiazolyl, oxazolyl or furyl or is phenyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
   and to the plant-compatible acid addition salts or metal salts thereof.

Furthermore, the invention relates to the use of the compounds of the formula I for controlling phytopathogenic fungi and to compositions comprising these compounds.

Azolylmethyloxiranes, their preparation and their use in crop protection are known, for example, from EP-A 0 094 564 and EP-A 0 196 038.

Azolylmethyloxiranes which carry a hetaryl substituent on the oxirane ring are known from EP-A 0 421 125.

Although the azolylmethyloxiranes described already have good to very good fungicidal activity against a number of pathogens, it was the object of the present invention to provide novel azolylmethyloxiranes having improved fungicidal activity.

This object was achieved with the compounds of the formula I described at the outset.

Owing to the basic character of their nitrogen atoms, the compound I is capable of forming salts or adducts with inorganic or organic acids or with metal ions.

Examples of inorganic acids are hydrohalic acids, such as hydrogen fluoride, hydrogen chloride, hydrogen bromide and hydrogen iodide, carbonic acid, sulfuric acid, phosphoric acid and nitric acid.

Suitable organic acids are, for example, formic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphoric acid radicals), where the alkyl or aryl radicals may carry further substituents, for example p-toluenesulfonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminum, tin and lead and also of the elements of transition groups one to eight, in particular chromium, manganese, iron, cobalt, nickel, copper, zinc, and others. Particular preference is given to the metal ions of the elements of transition groups of the fourth period. The metals can be present in the various valencies that they can assume.

The preparation of the compounds of the formulae is known and described in detail in EP-A 0 094 564, EP-A 0 196 038 and EP-A 0 421 125.

In the definitions of the symbols given in the formulae above, collective terms have been used which are generally representative of the substituents below:

Halogen: fluorine, chlorine, bromine and iodine;

Alkyl and the alkyl moieties of composite groups such as, for example, alkylamino: such as saturated straight-chain or branched hydrocarbon radicals having preferably 1 to 4 carbon atoms, such as methyl, ethyl, propyl, 1-methylethyl, butyl, 1-methylpropyl, 2-methylpropyl and 1,1-dimethylethyl.

Haloalkyl: alkyl as mentioned above, where some or all of the hydrogen atoms in these groups are replaced by halogen atoms as mentioned above. In one embodiment, the alkyl groups are substituted at least once or completely by a particular halogen atom, preferably fluorine, chlorine or bromine. In a further embodiment, the alkyl groups are partially or fully halogenated by different halogen atoms; in the case of mixed halogen substitutions, the combination of chlorine and fluorine is preferred. Particular preference is given to (C₁-C₄)-haloalkyl, more preferably (C₁-C₂)-haloalkyl, such as chloromethyl, bromomethyl, dichloromethyl, trichloromethyl, fluoromethyl, difluoro-methyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

Alkoxy: an alkyl group as defined above which is attached via an oxygen, preferably having 1 to 4 carbon atoms. Examples of preferred alkoxy groups are: methoxy, ethoxy, n-propoxy, 1-methylethoxy, butoxy, 1-methylpropoxy, 2-methylpropoxy or 1,1-dimethylethoxy.

Haloalkoxy: alkoxy as defined above, where some or all of the hydrogen atoms in these groups are replaced by halogen atoms as described above under haloalkyl, in particular fluorine, chlorine or bromine. Examples of preferred haloalkoxy radicals are OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy.

Alkylthio: alkyl as defined above which is attached via a sulfur atom.

The novel compounds of the formula I contain chiral centers and are generally obtained in the form of racemates or as diastereomer mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers. Suitable for use as antimicrobial agents are both the uniform diastereomers or enantiomers and mixtures thereof obtained in the synthesis. This applies correspondingly to the fungicidal compositions.

Preference is given here to pairs of enantiomers or enantiomers with cis arrangement of ring B and triazolylmethyl substituent.

The compounds according to the invention may be present in various crystal modifications which may differ in their biological activity. They are likewise provided by the present invention.

In the compounds of the formula I according to the invention or in the compounds of the formula I used according to the invention, the following meanings of the substituents, in each case on their own or in combination, are particularly preferred. Here, the preferred substituents or preferred combinations of substituents apply, if appropriate, correspondingly to the precursors of the compounds according to the invention.

The substituent A is phenyl which is substituted by three F, such that in total six substituents A1 to A6 of the following formulae result:

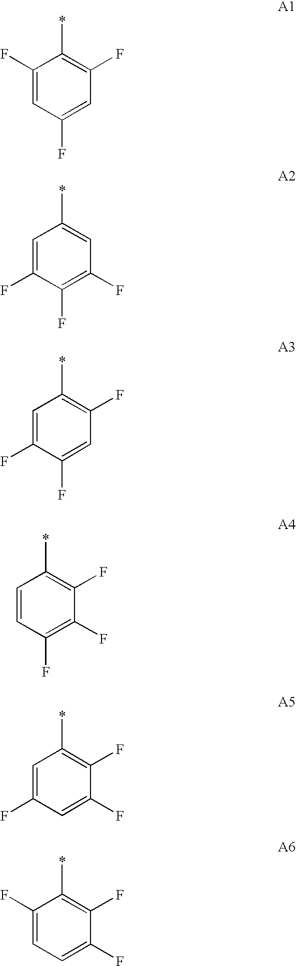

According to one embodiment, A is A1, A2, A3 or A4.

According to a further embodiment, A is A1 or A2.

According to a preferred embodiment, A is A1.

According to a further embodiment, A is A2.

According to a further embodiment, A is A3.

According to a further embodiment, A is A4.

According to another embodiment, A is A5 or A6.

According to a further embodiment, A is A5.

According to a further embodiment, A is A6.

A further embodiment relates to compounds I in which B is unsubstituted pyridyl, thienyl, thiazolyl, oxazolyl or furyl.

According to a preferred embodiment, B is pyridyl or thienyl.

According to a further preferred embodiment, B is pyridyl.

In a further embodiment of the present invention, B is phenyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio.

In a further embodiment, B is phenyl which is substituted by one to three of the following substituents: halogen, C₁-C₄-alkyl, C₁-C₄-alkoxy, C₁-C₄-haloalkyl or C₁-C₄-haloalkoxy.

In a preferred embodiment, B is phenyl which is substituted by one to three halogens.

In particular with a view to their use, preference is given to the compounds I according to the invention compiled in Tables 2 to 7 below. The groups mentioned for a substituent in the tables are furthermore per se, independently of the combination in which they are mentioned, a particularly preferred embodiment of the substituent in question.

TABLE 1

| Row | Substituent B |
|---|---|
| 1-1 | 2-methylphenyl |
| 1-2 | 3-methylphenyl |
| 1-3 | 4-methylphenyl |
| 1-4 | 2-methoxyphenyl |
| 1-5 | 3-methoxyphenyl |
| 1-6 | 4-methoxyphenyl |
| 1-7 | 2-chlorophenyl |
| 1-8 | 3-chlorophenyl |
| 1-9 | 4-chlorophenyl |
| 1-10 | 2-fluorophenyl |
| 1-11 | 3-fluorophenyl |
| 1-12 | 4-fluorophenyl |
| 1-13 | 2-chloro-3-methoxyphenyl |
| 1-14 | 2-chloro-4-methoxyphenyl |
| 1-15 | 2,3-dichlorophenyl |
| 1-16 | 2,4-dichlorophenyl |
| 1-17 | 3,4-dichlorophenyl |
| 1-18 | 2,3-difluorophenyl |
| 1-19 | 2,4-difluorophenyl |
| 1-20 | 2-chloro-3-fluorophenyl |
| 1-21 | 2-chloro-4-fluorophenyl |
| 1-22 | 2-pyridyl |
| 1-23 | 3-pyridyl |
| 1-24 | 4-pyridyl |
| 1-25 | 2-thienyl |
| 1-26 | 3-thienyl |
| 1-27 | thiazol-4-yl |
| 1-28 | thiazol-5-yl |
| 1-29 | oxazol-4-yl |
| 1-30 | oxazol-5-yl |
| 1-31 | 2-furyl |

### Table 2

Compounds of the formula I in which A is A1 and B corresponds in each case to a substituent of a row of Table 1.

### Table 3

Compounds of the formula I in which A is A2 and B corresponds in each case to a substituent of a row of Table 1.

### Table 4

Compounds of the formula I in which A is A3 and B corresponds in each case to a substituent of a row of Table 1.

### Table 5

Compounds of the formula I in which A is A4 and B corresponds in each case to a substituent of a row of Table 1.

### Table 6

Compounds of the formula I in which A is A5 and B corresponds in each case to a substituent of a row of Table 1.

### Table 7

Compounds of the formula I in which A is A6 and B corresponds in each case to a substituent of a row of Table 1.

The compounds I are suitable as fungicides. They are distinguished by an excellent activity against a broad spectrum of phytopathogenic fungi from the class of the Ascomycetes, Deuteromycetes, Oomycetes and Basidiomycetes, in particular from the class of the Oomycetes. Some of them are systemically effective and can be used in crop protection as foliar fungicides, as fungicides for seed dressing and as soil fungicides.

They are particularly important in the control of a multitude of fungi on various crop plants, such as wheat, rye, barley, oats, rice, corn, grass, bananas, cotton, soya, coffee, sugar cane, vines, fruit and ornamental plants, and vegetable plants, such as cucumbers, beans, tomatoes, potatoes and cucurbits, and on the seeds of these plants.

They are especially suitable for controlling the following plant diseases:
Alternaria species on vegetables, oilseed rape, sugar beet and fruit and rice, such as, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns, such as, for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochliobolus species on corn, cereals, rice, such as, for example, Cochliobolus sativus on cereals, Cochliobolus miyabeanus on rice;
Colletotricum species on soybeans and cotton;
Drechslera species, Pyrenophora species on corn, cereals, rice and lawns, such as, for example, D. teres on barley or D. tritici-repentis on wheat;
Esca on grapevines, caused by Phaeoacremonium chlamydosporium, Ph. Aleophilum and Formitipora punctata (syn. Phellinus punctatus);
Exserohilum species on corn;
Erysiphe cichoracearum and Sphaerotheca fuliginea on cucumber plants;
Fusarium and Verticillium species on various plants, such as, for example, F. graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and groundnuts, such as, for example, M. graminicola on wheat or M. fijiensis on bananas;
Peronospora species on cabbage and bulbous plants, such as, for example, P. brassicae on cabbage or P. destructor on onion;
Phakopsara pachyrhizi and Phakopsara meibomiae on soybeans;
Phomopsis species on soybeans and sunflowers;
Phytophthora infestans on potatoes and tomatoes;
Phytophthora species on various plants, such as, for example, P. capsici on bell pepper;
Plasmopara viticola on grapevines;
Podosphaera leucotricha on apple;

Pseudocercosporella herpotrichoides on cereals;
Pseudoperonospora on various plants, such as, for example, P. cubensis on cucumber or P. humili on hops;
Puccinia species on various plants, such as, for example, P. triticina, P. striformins, P. hordei or P. graminis on cereals or P. asparagi on asparagus;
Pyricularia oryzae, Corticium sasakii, Sarocladium oryzae, S. attenuatum, Entyloma oryzae on rice;
Pyricularia grisea on lawns and cereals;
Pythium spp. on lawns, rice, corn, cotton, oilseed rape, sunflowers, sugar beet, vegetables and other plants, such as, for example, P. ultiumum on various plants, P. aphanidermatum on lawns;
Rhizoctonia species on cotton, rice, potatoes, lawns, corn, oilseed rape, potatoes, sugar beet, vegetables and on various plants, such as, for example, R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, such as, for example, U. maydis on corn;
Venturia species (scab) on apples and pears, such as, for example, V. inaequalis on apple.

They are particularly suitable for controlling harmful fungi from the class of the Peronosporomycetes (syn. Oomycetes), such as Peronospora species, Phytophthora species, Plasmopara viticola, Pseudoperonospora species and Pythium species.

The compounds I are also suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds or materials to be protected against fungal attack or the soil with a fungicidally effective amount of the active compounds. Application can be both before and after the infection of the materials, plants or seeds by the fungi.

The fungicidal compositions generally comprise between 0.1 and 95% by weight, preferably between 0.5 and 90% by weight, of active compound.

When employed in crop protection, the application rates are, depending on the kind of effect desired, between 0.01 and 2.0 kg of active compound per ha.

In seed treatment, the amounts of active compound required are generally from 1 to 1000 g/100 kg of seed, preferably from 5 to 100 g/100 kg of seed.

When used in the protection of materials or stored products, the active compound application rates depend on the kind of application area and on the desired effect. Amounts typically applied in the protection of materials are, for example, from 0.001 g to 2 kg, preferably from 0.005 g to 1 kg, of active compound per cubic meter of treated material.

The compounds of the formula I can be present in different crystal modifications which may differ in their biological activity. They are likewise subject matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example finely divided silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a formulation having an active compound content of 10% by weight.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight.

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

2. Products to be Applied Undiluted

### I Dusts (DP, DS)

5 parts by weight of the active compounds are ground finely and mixed intimately with 95 parts by weight of finely divided kaolin. This gives a dustable product with an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

### K ULV Solutions (UL)

10 parts by weight of the active compounds are dissolved in 90 parts by weight of an organic solvent, for example xylene. This gives a product with an active compound content of 10% by weight to be applied undiluted.

Water-soluble concentrates (LS), suspensions (FS), dusts (DS), water-dispersible and water-soluble powders (WS, SS), emulsions (ES), emulsifiable concentrates (EC) and gel formulations (GF) are usually used for the treatment of seed. These formulations can be applied to the seed in undiluted or, preferably, diluted form. The application can be carried out before sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention in the application form as fungicides can also be present together with other active compounds, for example with herbicides, insecticides, growth regulators, fungicides or else with fertilizers. When mixing the compounds I or the compositions comprising them with one or more further active compounds, in particular fungicides, it is in many cases possible, for example, to widen the activity spectrum or to prevent the development of resistance. In many cases, synergistic effects are obtained.

The present invention furthermore provides a combination of at least one azolylmethyloxirane of the formula I, in particular an azolylmethyloxirane disclosed in the present description as being preferred, and/or an agriculturally acceptable acid addition salt or metal salt thereof and at least one further fungicidal, insecticidal, herbicidal and/or growth-regulating active compound, it being also possible for synergistic effects to occur.

The present invention also provides a pesticidal composition which comprises at least one compound of the formula I, in particular a compound of the formula I described in the present description as being preferred, and/or an agriculturally acceptable acid addition salt or metal salt thereof and at least one solid or liquid carrier. Such a pesticidal composition may comprise at least one further fungicidally, insecticidally and/or herbicidally active compound, it also being possible for a synergistic effect to occur.

The following list L of fungicides with which the compounds according to the invention can be applied together is meant to illustrate the possible combinations, but not to limit them:

### List L:

strobilurins
azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethyl-phenyloxymethylene)phenyl)-3-methoxyacrylate;

### L-2

carboxamides
carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoro-methyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloro-isothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chloro-phenyl)prop-2-ynyloxy]-3-methoxyphenyl)ethyl)-2-methanesulfonylamino-3-methyl-butyramide, N-(2-(4-[3-(4-chlorophenyl)prop-2-ynyloxy]-3-methoxyphenyl)ethyl)-2-ethanesulfonylamino-3-methylbutyramide;
   azoles
triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;
   nitrogenous heterocyclyl compounds
pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propyl-chromen-4-one, N,N-dimethyl-3-(3-bromo-6-fluoro-2-methylindole-1-sulfonyl)-[1,2,4]triazole-1-sulfonamide;
   carbamates and dithiocarbamates
dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;
   other fungicides
guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### SYNTHESIS EXAMPLES

### Example 1

### 1) Synthesis of 2-chloro-1-(2,4,5-trifluorophenyl)ethanone

N-chlorosuccinimide (11.9 g, 89.3 mmol) was added to a solution of 2,4,5-trifluoro-acetophenone (14.9 g, 85.0 mmol) in acetic acid (150 ml). The reaction mixture was stirred at 116° C. for five hours, cooled to room temperature and neutralized with aqueous sodium hydroxide solution. The aqueous phase was extracted with ethyl acetate (2×40 ml). The combined organic phases were dried over sodium sulfate, and the solvent was distilled off. The residue was purified by column chromatography (silica gel, hexane/dichloromethane 7:3). The appropriate fractions were combined, giving 2-chloro-1-(2,4,5-trifluorophenyl)ethanone (9.5 g, 54%) in the form of a white solid. H-NMR (300 MHz, CDCl₃): δ 7.88-7.79 (m, 1H), 7.11-7.02 (m, 1H), 4.96 (d, J=3.2 Hz, 2H).

### 2) Synthesis of 1-chloro-2-(2,4,5-trifluorophenyl)-3-(4-fluorophenyl)propan-2-ol

Magnesium turnings (415 mg, 16.5 mmol) and iodine were added to a solution of 4-fluorobenzyl chloride (238 mg, 1.64 mmol) in anhydrous diethyl ether (20 ml). The reaction mixture was warmed slowly until the iodine color disappeared and the start of the reaction was indicated by the refluxing solvent. The remaining 4-fluorobenzyl chloride (2.14 g, 11.7 mmol) was added dropwise such that the reaction mixture was kept at reflux. After the addition had ended, the mixture was stirred at room temperature for another two hours and then cooled to 0° C. At this temperature, 2-chloro-1-(2,4,5-trifluorophenyl)ethanone (3.1 g, 14.8 mmol) in anhydrous toluene (10 ml) was added dropwise. The mixture was then warmed to room temperature and stirred for another three hours. After this time, the mixture was again cooled to 0° C., and a saturated aqueous ammonium chloride solution (10 ml) was added. The organic phase was removed and the aqueous phase was extracted with ethyl acetate (2×20 ml). The combined organic extracts were dried over sodium sulfate and freed from the solvent. The crude product obtained in this manner (5.0 g) was used without purification for the next reaction step.

### 3) Synthesis of (Z)-1-[3-chloro-1-(4-fluorophenyl)prop-1-en-2yl]-2,4,5-tritluorobenzene

At 0° C., first acetic anhydride (1.8 ml, 14.8 mmol) and then concentrated sulfuric acid (0.1 ml, 1.90 mmol) were added to a solution of 1-chloro-2-(2,4,5-trifluorophenyl)-3-(4-fluorophenyl)propan-2-ol (5.0 g, approximately 15.7 mmol) in a 1,4-dioxane/THF mixture (44 ml, 10:1). The reaction mixture was then warmed to room temperature, stirred for another 18 h and subsequently cooled again to 0° C. At this temperature, saturated sodium chloride solution (20 ml) was added, and the mixture was neutralized using aqueous sodium hydroxide solution (6.2 ml, 50% w/w). The resulting mixture was extracted with ethyl acetate (3×20 ml), and the organic phases were combined, washed with water (2×30 ml) and dried over sodium sulfate. After filtration and removal of the solvent under reduced pressure, the residue was purified by column chromatography (silica gel, hexane). The appropriate fractions were combined, giving (Z)-1-[3-chloro-1-(4-fluorophenyl)prop-1-en-2-yl]-2,4,5-trifluorobenzene in the form of a colorless oil (490 mg, 10% over 2 steps).

¹H NMR (300 MHz, CDCl₃): δ7.45-7.41 (m, 2H), 7.16-7.10 (m, 2H), 7.00-6.92 (m, 2H), 6.74 (s, 1H), 4.51 (s, 2H).

### 4) Synthesis of anti-2-(2,4,5-trifluorophenyl)-2-(chloromethyl)-3-(4-fluorophenyl)oxirane

Maleic anhydride (1.5 g, 16.3 mmol) and aqueous hydrogen peroxide solution (0.5 ml of a 50% strength solution, 16.3 mmol) were added to a solution of (Z)-1-[3-chloro-1-(4-fluorophenyl)prop-1-en-2-yl]-2,4,5-trifluorobenzene (490 mg, 1.6 mmol) in acetic acid (20 ml). The reaction mixture was stirred at 40° C. for 18 h and then cooled to room temperature, and water (20 ml) and aqueous sodium thiosulfate solution (10% strength solution, 4 ml) were added. The aqueous phase was extracted with dichloromethane (3×15 ml), and the combined organic phases were washed with sodium chloride solution (2×10 ml). The organic phase was dried over sodium sulfate and filtered off, and the solvent was distilled off. The residue obtained in this manner was purified by column chromatography (silica gel, 25:1 hexane/ethyl acetate). The appropriate fractions were combined, giving the target compound (320 mg, 62%) in the form of a colorless solid.

¹H NMR (300 MHz, CDCl₃): δ 7.44-7.35 (m, 3H), 7.13 (t, J=8.6 Hz, 2H), 7.03-6.95 (m, 1H), 4.20 (s, 1H), 3.78 (d, J=12.0 Hz, 1H), 3.37 (d, J=12.0 Hz, 1H).

### 5) Synthesis of 1-[(anti)-2-(2,4,5-trifluorophenyl)-3-(4-fluorophenyl)oxiran-2-yl)methyl]-1H-1,2,4-triazole

At room temperature, 1,2,4-triazole (209 mg, 3.0 mmol) and sodium hydride (75 mg, 3.1 mmol) were added to a solution of anti-2-(2,4,5-trifluorophenyl)-2-(chloromethyl)-3-(4-fluorophenyl)oxirane (320 mg, 1.0 mmol) in anhydrous N,N-dimethylformamide (20 ml). The mixture was stirred at 75° C. for 18 hours. The mixture was then cooled to room temperature, diluted with ethyl acetate (20 ml) and washed with sodium chloride solution (3×15 ml). The organic phase was removed, dried over sodium sulfate and filtered, and the solvent was distilled off. The residue obtained in this manner was purified by column chromatography (silica gel, 7:3 hexane/ethyl acetate). The appropriate fractions were combined, giving the target compound (121 mg, 35%) in the form of a colorless solid.

¹H NMR (300 MHz, acetone-d₆): δ 7.89 (s, 1H), 7.82 (s, 1H), 7.52-7.48 (m, 2H), 7.18 (t, J=8.0 Hz, 2H), 7.09-6.93 (m, 2H), 4.64 (d, J=15.0 Hz, 1H), 4.21 (s, 1H), 4.04 (d, J=15.0 Hz, 1H).

### Example 2

### 1) Synthesis of 2-chloro-1-(3,4,5-trifluorophenyl)ethanone

N-chlorosuccinimide (11.9 g, 89.1 mmol) was added to a solution of 3,4,5-trifluoro-acetophenone (14.9 g, 85.6 mmol) in acetic acid (150 ml). The reaction mixture was stirred at 116° C. for 4.5 hours, cooled to room temperature and neutralized with aqueous sodium hydroxide solution. The aqueous phase was extracted with ethyl acetate (2×40 ml). The combined organic phases were dried over sodium sulfate, and the solvent was distilled off. The residue was purified by column chromatography (silica gel, hexane/dichloromethane 7:3). The appropriate fractions were combined, giving 2-chloro-1-(3,4,5-trifluorophenyl)ethanone (9.2 g, 53%) in the form of a white solid. H-NMR (300 MHz, CDCl₃): δ 7.66-7.61 (m, 2H), 4.57 (s, 2H).

### 2) Synthesis of 1-chloro-2-(3,4,5-trifluorophenyl)-3-(4-fluorophenyl)propan-2-ol

Magnesium turnings (398 mg, 16.6 mmol) and iodine were added to a solution of 4-fluorobenzyl chloride (250 mg, 1.55 mmol) in anhydrous diethyl ether (25 ml). The reaction mixture was warmed slowly until the iodine color disappeared and the start of the reaction was indicated by the refluxing solvent. The remaining 4-fluorobenzyl chloride (2.80 g, 13.5 mmol) was added dropwise such that the reaction mixture was kept at reflux. After the addition had ended, the mixture was stirred at room temperature for another two hours and then cooled to 0° C. At this temperature, 2-chloro-1-(3,4,5-trifluorophenyl)ethanone (2.36 g, 14.3 mmol) in anhydrous toluene (10 ml) was added dropwise. The mixture was then warmed to room temperature and stirred for three hours. After this time, the mixture was again cooled to 0° C., and a saturated aqueous ammonium chloride solution (10 ml) was added. The organic phase was separated off, and the aqueous phase was extracted with ethyl acetate (2×20 ml). The combined organic extracts were dried over sodium sulfate and freed from the solvent. The crude product obtained in this manner (5.0 g) was used without purification for the next reaction step.

### 3) Synthesis of (Z)-1-[3-chloro-1-(4-fluorophenyl)prop-1-en-2-yl]-3,4,5-trifluorobenzene

At 0° C., first acetic anhydride (1.8 ml, 14.8 mmol) and then concentrated sulfuric acid (0.1 ml. 1.90 mmol) were added to a solution of 1-chloro-2-(3,4,5-trifluorophenyl)-3-(4-fluorophenyl)propan-2-ol (5.0 g, approximately 15.7 mmol) in a 1,4-dioxane/THF mixture (44 ml, 10:1). The reaction mixture was then warmed to room temperature, stirred for another 18 h and subsequently cooled again to 0° C. At this temperature, saturated sodium chloride solution (20 ml) was added, and the mixture was neutralized with aqueous sodium hydroxide solution (6.2 ml. 50% w/w). The mixture was extracted with ethyl acetate (3×20 ml), and the organic phases were combined, washed with water (2×30 ml) and dried over sodium sulfate. After filtration and removal of the solvent under reduced pressure, the residue was purified by column chromatography (silica gel, hexane). The appropriate fractions were combined, giving (Z)-1-[3-chloro-1-(4-fluorophenyl)prop-1-en-2-yl]-3,4,5-trifluorobenzene in the form of a colorless oil (814 mg, 19% over 2 steps).

¹H NMR (300 MHz, CDCl₃): δ 7.62-7.59 (m, 1H), 7.47-7.44 (m, 1H), 7.37-7.32 (m, 2H), 7.27-7.22 (m, 2H), 7.05 (s, 1H), 4.42 (s, 2H).

### 4) Synthesis of anti-2-(3,4,5-trifluorophenyl)-2-(chloromethyl)-3-(4-fluorophenyl)oxirane

Maleic anhydride (554 mg, 5.6 mmol) and aqueous hydrogen peroxide solution (0.1 ml of a 50% strength solution, 5.6 mmol) were added to a solution of (Z)-1-[3-chloro-1-(4-fluorophenyl)prop-1-en-2-yl]-3,4,5-trifluorobenzene (170 mg, 0.5 mmol) in acetic acid (10 ml). The reaction mixture was stirred at 40° C. for 18 h and then cooled to room temperature, and water (20 ml) and aqueous sodium thiosulfate solution (10% strength solution, 4 ml) were added. The aqueous phase was extracted with dichloromethane (3×15 ml), and the combined organic phases were washed with sodium chloride solution (2×10 ml). The organic phase was dried over sodium sulfate and filtered off, and the solvent was distilled off.

The residue obtained in this manner was purified by column chromatography (silica gel, 25:1 hexane/ethyl acetate). The appropriate fractions were combined, giving anti-2-(3,4,5-trifluorophenyl)-2-(chloromethyl)-3-(4-fluorophenyl)oxirane (135 mg, 75%) in the form of a colorless solid.

¹H NMR (300 MHz, CDCl₃): δ 7.40-7.35 (m, 2H), 7.19-7.09 (m, 4H), 4.09 (s, 1H), 3.76 (d, J=12.0 Hz, 1H), 3.53 (d, =12.0 Hz, 1H).

### 5) Synthesis of 1-[(anti)-2-(3,4,5-trifluorophenyl)-3-(4-fluorophenyl)oxiran-2-yl)methyl]-1H-1,2,4-triazole

At room temperature, 1,2,4-triazole (88 mg, 1.2 mmol) and sodium hydride (31 mg, 1.3 mmol) were added to a solution of anti-2-(3,4,5-trifluorophenyl)-2-(chloromethyl)-3-(4-fluorophenyl)oxirane (135 mg, 0.4 mmol) in anhydrous N,N-dimethylformamide (10 ml). The mixture was stirred at 50° C. for 18 hours. The mixture was then cooled to room temperature, diluted with ethyl acetate (20 ml) and washed with sodium chloride solution (3×15 ml). The organic phase was removed, dried over sodium sulfate and filtered, and the solvent was distilled off. The residue obtained in this manner was purified by column chromatography (silica gel, 7:3 hexane/ethyl acetate, then silica gel, 9:1 dichloromethane/ethyl acetate). The appropriate fractions were combined, giving the target compound (33 mg, 22%) in the form of a colorless solid.

¹H NMR (300 MHz, acetone-d₆): δ 7.91 (s, 1H), 7.84 (s, 1H), 7.47-7.42 (m, 2H), 7.16 (t, J=9.0 Hz, 2H), 7.06-7.01 (m, 2H), 4.68 (d, J=15.0 Hz, 1H), 4.12 (s, 1H), 4.13 (d, J=15.0 Hz, 1H).

Analogously to this preparation process, the compounds of the general formula I of Table 8 were prepared as racemates having a cis arrangement of triazolylmethyl substituent and ring B.

TABLE 8

| | | | Physical data |
|---|---|---|---|
| Comp. | | | FP [° C.], ¹H-NMR |
| No. | A | B | (300 MHz, CDC13) |
| 8.1 | 3,4,5-trifluorophenyl | 2-methylphenyl | 7.81 (2H), 7.46 (1H), |
| | | | 7.33 (2H), 7.23 (1H), |
| | | | 7.10 (2H), 4.67 (1H), |
| | | | 4.09 (1H), 4.06 (1H), |
| | | | 2.30 (3H) |
| 8.2 | 3,4,5-trifluorophenyl | 2-chlorophenyl | 135-139 |
| 8.3 | 3,4,5-trifluorophenyl | 2-fluorophenyl | 118-120 |
| 8.4 | 3,4,5-trifluorophenyl | 4-fluorophenyl | 103-105 |
| 8.5 | 2,4,5-trifluorophenyl | 4-fluorophenyl | 140-143 |
| 8.6 | 2,4,5-trifluorophenyl | 2-chlorophenyl | 134-136 |
| 8.7 | 2,4,5-trifluorophenyl | 2-methylphenyl | 7.91 (1H), 7.79 (1H), |
| | | | 7.51 (1H), 7.31 (3H), |
| | | | 7.05 (2H), 4.73 (1H), |
| | | | 4.18 (1H), 3.99 (1H), |
| | | | 2.44 (3H) |

### Biological Tests

### Greenhouse

### Preparation of Active Compound

The active compounds were prepared separately or jointly as a stock solution comprising 25 mg of active compound which was made up to 10 ml using a mixture of acetone and/or DMSO and the emulsifier Wettol EM 31 (wetting agent having emulsifying and dispersing action based on ethoxylated alkylphenols) in a volume ratio of solvent/emulsifier of 99:1. The mixture was then made up to 100 ml with water. This stock solution was diluted with the solvent/emulsifier/water mixture described to give the concentration of active compounds stated below.

### Use Example 1

### Protective Activity Against Puccinia recondite on Wheat (Brown Rust of Wheat) (Puccrt P1)

Leaves of potted wheat seedlings were sprayed to runoff point with an aqueous suspension having the active compound concentration stated below. The next day, the treated plants were inoculated with a spore suspension of brown rust of wheat (Puccinia recondite). The plants were then placed in a chamber with high atmospheric humidity (90 to 95%) at 20 to 22° C. for 24 hours. During this time, the spores germinated and the germ tubes penetrated into the leaf tissue. The next day, the test plants were returned to the greenhouse and cultivated at temperatures between 20 and 22° C. and at 65 to 70% relative atmospheric humidity for a further 7 days. The extent of the rust fungus development on the leaves was then determined visually.

The plants which had been treated with an aqueous active compound preparation comprising 63 ppm of the active compound 8.5, 8.6, 8.7, 8.1 or 8.2 of Table 8 showed an infection of at most 10%, whereas the untreated plants were 90% infected.

### Use Example 2

### Activity Against Mildew on Cucumber Leaves Caused by Sphaerotheca fuliginea, Protective Application (Sphrfu P1)

Leaves of potted cucumber seedlings were, at the cotyledon stage, sprayed to runoff point with an aqueous suspension having the active compound concentration stated below. 20 hours after the spray coating had dried on, the plants were inoculated with an aqueous spore suspension of mildew of cucumber (Sphaerotheca fuliginea). The plants were then cultivated in a greenhouse at temperatures between 20 and 24° C. and 60 to 80% relative atmospheric humidity for 7 days. The extent of the mildew development was then determined visually in % infection of the cotyledon area.

The plants which had been treated with an aqueous active compound preparation comprising 63 ppm of the active compound 8.6, 8.7, 8.1 and 8.2 of Table 8 showed an infection of at most 7%, whereas the untreated plants were 90% infected.

### Comparative Tests

### Greenhouse

### Preparation of Active Compound

The active compounds were prepared separately or jointly as a stock solution comprising 25 mg of active compound which was made up to 10 ml using a mixture of acetone and/or DMSO and the emulsifier Wettol EM 31 (wetting agent having emulsifying and dispersing action based on ethoxylated alkylphenols) in a volume ratio of solvent/emulsifier of 99:1. The mixture was then made up to 100 ml with water. This stock solution was diluted with the solvent/emulsifier/water mixture described to give the concentration of active compounds stated below.

### Comparative Example 1

### Activity Against Mildew on Cucumber Leaves Caused by Sphaerotheca fuliginea, Protective Application (Sphrfu P1)

Leaves of potted cucumber seedlings were, at the cotyledon stage, sprayed to runoff point with an aqueous suspension having the active compound concentration stated below. 20 hours after the spray coating had dried on, the plants were inoculated with an aqueous spore suspension of mildew of cucumber (Sphaerotheca fuliginea). The plants were then cultivated in a greenhouse at temperatures between 20 and 24° C. and 60 to 80% relative atmospheric humidity for 7 days. The extent of the mildew development was then determined visually in % infection of the cotyledon area.

TABLE 9

| | | Infection |
|---|---|---|
| | | in % at |
| | | 63 ppm |
| Comp. | | for Sphrfu |
| No.: | Structure | P1 |
| 8.7 | 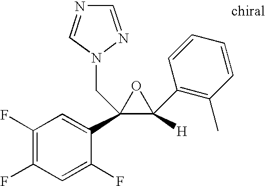 | 0 |
| B 255 from EP-A 0 421 125 | 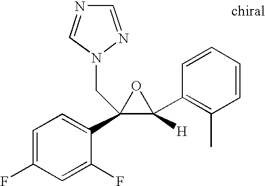 | 90 |
| untreated | | 90 |

As can be seen from the values of Table 9, the compound No. 8.7 according to the invention has considerably better fungicidal activity than the structurally most similar compound B 255 of the prior art from EP-A 0 421 125.

### Comparative Example 2

### Activity Against Net Blotch of Barley Caused by Pyrenophora teres, 1 Day Protective Application (Pymte P1)

Leaves of potted barley seedlings were sprayed to runoff point with an aqueous suspension having the active compound concentration stated below. 24 hours after the spray coating had dried on, the test plants were inoculated with an aqueous spore suspension of Pyrenophora[syn. Drechslera] teres, the net blotch pathogen. The test plants were then placed in a greenhouse at temperatures between 20 and 24° C. and at 95 to 100% relative atmospheric humidity. After 6 days, the extent of the development of the disease was determined visually in % infection of the entire leaf area.

TABLE 10

| | | Infection |
|---|---|---|
| | | in % at |
| | | 4 ppm |
| Comp. | | for Pyrnte |
| No.: | Structure | P1 |
| 8.6 | 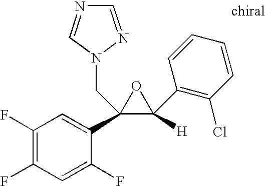 | 40 |
| Comp. 8 of EP-A 0 196 038 | 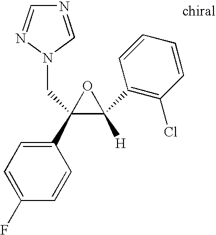 | 90 |
| untreated | | 90 |

As can be seen from the values of Table 10, the compound No. 8.6 according to the invention shows considerably better fungicidal activity than the structurally most similar compound No. 8 of the prior art from EP-A 0 196 038.

1-9. (canceled)

10. An azolylmethyloxirane of the general formula I 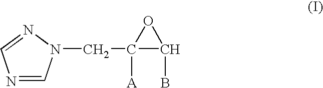
wherein
A is phenyl which is substituted by three F,
B is unsubstituted pyridyl, thienyl, thiazolyl, oxazolyl or furyl or is phenyl which is substituted by one to three of the following substituents: halogen, NO₂, amino, C₁-C₁-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
or a plant-compatible acid addition salt or metal salt thereof.

11. The compound according to claim 10 wherein A is 2,4,6-trifluorophenyl, 3,4,5-trifluorophenyl or 2,4,5-trifluorophenyl.

12. The compound according to claim 10 wherein B is pyridyl, thienyl, thiazolyl, oxazolyl or furyl.

13. The compound according to claim 10 wherein B is phenyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio.

14. The compound according to claim 13 wherein B is phenyl which is substituted by one to three of the following substituents: halogen, C₁-C₄-alkyl, C₁-C₄-alkoxy, C₁-C₄-haloalkyl or C₁-C₄-haloalkoxy.

15. A crop protection composition comprising a solid or liquid carrier and the compound of claim 10 and/or an acid addition salt or metal salt thereof.

16. The composition of claim 15 wherein A is 2,4,6-trifluorophenyl, 3,4,5-trifluorophenyl or 2,4,5-trifluorophenyl.

17. The composition of claim 15 wherein B is pyridyl, thienyl, thiazolyl, oxazolyl or furyl.

18. The composition of claim 15 wherein B is phenyl which is substituted by one to three of the following substituents: halogen, NO₂, amino, C₁-C₄-alkyl, alkoxy, C₁-C₄-haloalkyl, C₁-C₄-haloalkoxy, C₁-C₄-alkylamino, C₁-C₄-dialkylamino, thio or C₁-C₄-alkylthio.

19. The composition of claim 18 wherein B is phenyl which is substituted by one to three of the following substituents: halogen, C₁-C₄-alkyl, C₁-C₄-alkoxy, C₁-C₄-haloalkyl or C₁-C₄-haloalkoxy.

20. A seed comprising the compound of claim 10 and/or an acid addition salt or metal salt thereof.

21. The seed of claim 20 wherein A is 2,4,6-trifluorophenyl, 3,4,5-trifluorophenyl or 2,4,5-trifluorophenyl.

22. The seed of claim 20 wherein B is pyridyl, thienyl, thiazolyl, oxazolyl or furyl.

23. The seed of claim 20 wherein B is phenyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio.

24. The seed of claim 23 wherein B is phenyl which is substituted by one to three of the following substituents: halogen, C₁-C₄-alkyl, C₁-C₄-alkoxy, C₁-C₄-haloalkyl or C₁-C₄-haloalkoxy.

25. A method for controlling phytopathogenic fungi wherein the fungi or the materials, plants, the soil or seed to be protected against fungal attack are/is treated with an effective amount of the compound of claim 10 or an acid addition salt or metal salt thereof.

26. The method of claim 25 wherein A is 2,4,6-trifluorophenyl, 3,4,5-trifluorophenyl or 2,4,5-trifluorophenyl.

27. The method of claim 25 wherein B is pyridyl, thienyl, thiazolyl, oxazolyl or furyl.

28. The method of claim 25 wherein B is phenyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio.

29. The method of claim 28 wherein B is phenyl which is substituted by one to three of the following substituents: halogen, C₁-C₄-alkyl, C₁-C₄-alkoxy, C₁-C₄-haloalkyl or C₁-C₄-haloalkoxy.
